(19) United States
(12) Patent Application Publication
(10) Pub. No.: US 2024/0350551 A1
(43) Pub. Date: Oct. 24, 2024

(54) TREATMENT OF AGING FRAILTY COMPRISING ADMINISTERING BONE MARROW DERIVED MESENCHYMAL STEM CELLS

(71) Applicant: Longeveron Inc., Miami, FL (US)

(72) Inventors: Joshua M. HARE, Miami, FL (US); Anthony A. OLIVA, Miami, FL (US); Ben HITCHINSON, Miami, FL (US)

(73) Assignee: Longeveron Inc., Miami, FL (US)

(21) Appl. No.: 18/682,949

(22) PCT Filed: Sep. 9, 2022
(86) PCT No.: PCT/US2022/043067

§ 371 Date: Feb. 12, 2024

Related U.S. Application Data
(60) Provisional application No. 63/261,092, filed on Sep. 10, 2021.

(51) Int. Cl. A61K 35/28; A61P 43/00; G01N 33/68

(52) U.S. Cl. CPC A61K 35/28; A61P 43/00; G01N 33/6869; G01N 2333/495; G01N 2333/525; G01N 2333/5406; G01N 2333/5421; G01N 2333/5428; G01N 2333/5434; G01N 2333/545; G01N 2333/55; G01N 2333/7155

(57) ABSTRACT

Compositions and methods are disclosed herein for the treatment of aging frailty with bone marrow derived mesenchymal stem cells. The methods of treatment involve the administration of a composition of bone marrow derived mesenchymal stem cells to a subject in need thereof, wherein the effectiveness of the treatment methods can be determined through the measurement of specific biomarkers and improved physical activity.

### CROSS-REFERENCE TO RELATED APPLICATIONS

This application claims benefit of U.S. Provisional Patent Application No. 63/261,092, filed on Sep. 10, 2021. That application is incorporated by reference as if fully rewritten herein.

### FIELD

The present disclosure relates to methods and compositions for the treatment of aging frailty in subjects in need thereof.

### BACKGROUND

Aging frailty poses a very concerning problem for the overall health and wellbeing of individuals. Aging frailty is a geriatric syndrome characterized by weakness, low physical activity, slowed motor performance, exhaustion, and unintentional weight loss. See Yao, X. et al., Clinics in Geriatric Medicine 27 (1): 79-87 (2011). Furthermore, there are many studies showing a direct correlation between aging frailty and inflammation. See Hubbard, R. E. et al., Biogerontology 11 (5): 635-641 (2010).

Immunosenescence is characterized by a low grade, chronic systemic inflammatory state known as inflammaging. See Franceshi, C. et al., Annals of the New York Academy of Sciences 908:244-254 (2000). This heightened inflammatory state or chronic inflammation found in aging and aging frailty leads to immune dysregulation and a complex remodeling of both innate and adaptive immunity. In immunosenescence, the T cell and B cell repertoire is skewed resulting in an increase in CD8⁺ T effector memory cells re-expressing CD45ra (TEMRA) and in the CD19⁺ late/exhausted memory B cells, and a decrease in the CD8⁺ Naive T cells, and in the switched memory B cells (CD27⁺). See Blomberg, B. B. et al., Immunologic Research 57 (1-3): 354-360 (2013); Colonna-Romano, G. et al., Mechanisms of Ageing and Development 130 (10): 681-690 (2009); and Koch S. et al., Immunity & Ageing: 5:6 (2008).

This shift in the T cell and B cell repertoire results in a refractory or less efficient immune status. This deterioration of the immune system causes greater susceptibility to infectious diseases and reduced responses to vaccination. Optimal B cell function is critical for production of effective antibody responses to vaccines and protection from infectious agents. It is well known that age-associated increase in systemic inflammation (TNF-a, IL-6, IL-8, INFy and CRP) induces impaired B cell function leading to poor antibody responses and decreased vaccine efficacy.

Inflammaging has received considerable attention because it proposes a link between immune changes and a number of diseases and conditions (such as aging frailty) common in old age. Circulating inflammatory mediators such as cytokines and acute phase proteins are markers of the low-grade inflammation observed to increase with aging. These pro-inflammatory cytokines (e.g., TNF-a, IL-6) impair the capacity of B cells to make protective antibodies to exogenous antigens and vaccines. This impaired B cell response is measured by reduced class switch recombination (CSR) which is the ability of immunoglobulins to switch isotype from IgM to a secondary isotype (IgG, IgA, or IgE). Immunoglobulin isotype switching is crucial for a proper immune response as the effector functions differ in each isotype. A key player in CSR and somatic hypermutation (SHM) is the enzyme, activation-induced cytidine deaminase (AID), encoded by the Aicda gene. AID's basic function in CSR and SHM is to initiate breaks in the DNA by converting cytosines to uracils in the switch and variable regions of immunoglobulins.

E47, encoded by the Tcfe2a (E2A) gene, is a transcription factor belonging to the class I basic helix loop helix (bHLH) proteins, also known as E proteins. Without E47 expression, the B cell specific transcription factors EBFI (early B cell factor) and Pax-5 (paired box protein) are not expressed. Both E47 and Pax-5 are key transcription factors in early development for the B cell lineage and mature B cell function. See Hagman J. et al., Immunity 27 (1): 8-10 (2007); Horcher M. et al., Immunity 14 (6): 779-790 (2001); Riley R.L. et al., Seminars in Immunology 17 (5): 330-336 (2005). The Pax-5 gene encodes the B cell lineage specific activator protein (BSAP) that is expressed at all stages of B cell differentiation, but not in terminally differentiated B cells. Pax-5 controls B cell commitment by repressing B lineage inappropriate genes and activating B cell specific genes making Pax-5 the B cell gatekeeper and is exclusively expressed in the B lymphoid lineage from the committed pro-B cell to the mature B cell stage. The B cell specific transcription factor, Pax-5, is not only highly important in early B cell development and B cell lineage commitment, it is also involved in CSR.

It has also been shown in humans that the amount of TNF-α made: (1) depends on the amount of system inflammation and (2) impairs the ability of the same B cells to be stimulated with mitogens or antigens. See Frasca, D. et al., Journal of Immunology 188 (1): 279-286 (2012). Thus, the immune response in subjects suffering from aging frailty is impaired for a number of reasons.

Aging frailty can impact a subject's quality of life in many ways. For example, aging frailty can lead to a decreased immune response after vaccination, thereby decreasing the effectiveness of the vaccine. Indeed, vaccination against influenza is strongly recommended in individuals over 65 years of age to protect them from infection. Although commercially available vaccines against influenza provide protection and ensure lasting immunological memory in children and adults, they are much less effective in elderly and frail individuals. See Frasca D. et al., Current Opinion in Immunology 29:112-118 (2014) and Yao X. et al., Vaccine 29 (31): 5015-5021 (2011). Despite receiving the influenza vaccine routinely, elderly individuals are at higher risk of infection with influenza leading to secondary complications, hospitalization, physical debilitation and ultimately death. See Gross, P. et al., Annals of Internal Medicine 123 (7): 518-527 (1995); Simonsen L. et al., The Journal of Infectious Diseases 178 (1): 53-60 (1998); and Vu T. et al., Vaccine 20 (13-14): 1831-1836 (2002).

These increased risks can be attributed to the development of aging frailty in this population of subjects. Influenza vaccines also prevent other complications that arise from influenza infection (e.g., pneumonia) in most elderly individuals, reducing the rate of hospitalization to some extent. Nichol K. L. et al., The New England Journal of Medicine 331 (12): 778-784 (1994). However, the rate of hospitalization due to influenza-related disease is still very high within this population. See Thompson, W. W. et al., JAMA 292 (11): 1333-1340 (2004).

Previous published results have reported that the specific response of B cells to the influenza vaccine in vitro (measured by AID), and the in vivo serum response (measured by HAI assay and ELISA), decrease with aging and are significantly correlated. See Frasca, D. et al., Vaccine 28 (51): 8077-8084 (2010). It was also reported that the percentage of switched memory B cells and CpG-induced AID, both measured before vaccination (t0), are decreased with aging and are significantly correlated with the in vivo response. Thus, these markers appear predictive of the in vivo response.

Therefore, in view of the above problems, the development of treatment methods and compositions that can improve the immune response and function of the immune system in the elderly would be beneficial for advancing the field of geriatric medicine.

### SUMMARY

An objective of the present disclosure is to provide methods of treatment or alleviation for aging frailty in subjects in need thereof wherein these methods comprise administering a therapeutic amount of bone marrow derived mesenchymal stem cells to a subject in need.

Another objective of the present disclosure is to provide novel biomarkers for diagnosing and evaluating the progression of aging frailty in a subject in need thereof. These novel biomarkers can also be measured to determine the effectiveness of the treatment methods described herein.

Some embodiments are drawn to compositions comprising a therapeutically effective amount of bone marrow derived stem cells, specifically bone marrow derived mesenchymal stem cells (bMSCs), which are used to alleviate the symptoms of aging frailty, such as a decreased immune response or an increase in systemic inflammation. Other embodiments are drawn to methods of treatment wherein subjects suffering from symptoms of aging frailty are administered compositions comprising a therapeutically effective amount of bMSCs. The effectiveness of these treatments can be evaluated through measuring the concentrations and expression of specific biomarkers, such as Tie2, VEGF and TGF-β, in subjects after administration of compositions comprising bMSCs.

### BRIEF DESCRIPTION OF DRAWINGS

FIG. 1 depicts a phase 2b, randomized, blinded and placebo-controlled clinical trial design for the evaluation of the safety and efficacy of Lomecel-B infusion in patients suffering from aging frailty.

FIG. 2A depicts a change in 6MWT versus time following treatment with Lomecel-B cells. All 4 Lomecel-B arms showed trending or significant increases in walk distance, in which the 200 M dose showed the greatest changes from baseline. In contrast, the placebo arm showed a trending decrease by 9 months post-treatment. *=p<0.05 for change from baseline. **=p<0.01 for change from baseline. #=p<0.05 for change from baseline of Lomecel-B arm versus change from baseline in placebo. ##=p<0.01 for change from baseline of Lomecel-B arm versus change from baseline in placebo.

FIG. 2B depicts the dose-response effect for each treatment group in FIG. 2A calculated using the MCP-Mod method. All five models were statistically significant, while the linear model yielded the smallest AIC value.

FIG. 3 depicts the modified intent-to-treat dose-response curves between the amount of Lomecel-B cells administered to patients and the change in their 6MWT scores.

FIG. 4A depicts the change in the PROMIS Physical Function Score at 6 months post-treatment, chosen as a secondary endpoint, significantly correlated to the change in 6MWT.

FIG. 4B depicts the change in the PROMIS Mobility at 6 months post-treatment, chosen as a secondary endpoint, significantly correlated to the change in 6MWT.

FIG. 4C depicts the change in the PROMIS Upper Extremity at 6 months post-treatment, chosen as a secondary endpoint, significantly correlated to the change in 6MWT.

FIG. 5A depicts the change in Soluble Tie2 (sTie2) after treatment with Lomecel-B cells. sTie2 decreased in the 200 M Lomecel-B group, which was significant at 3 months post-infusion. At 9 months post-infusion, the difference between the 200 M Lomecel-B group and placebo was highly significant. *=p<0.05 for change from baseline. ##=p<0.01 for change from baseline of Lomecel-B arm versus change from baseline in placebo.

FIG. 5B depicts the dose-response effect for each treatment group in FIG. 5A calculated using the MCP-Mod method, modeled with the Sigmoidal Emax (p=0.0175).

FIG. 5C depicts the inverse correlation between changes in sTie2 levels with changes in 6MWT.

FIG. 6A depicts the changes in TGF-B concentration after administration of 200 million bone marrow derived LOMECEL-B cells compared to placebo.

FIG. 6B depicts the changes in VEGF concentration after administration of 200 million bone marrow derived LOMECEL-B cells compared to placebo.

### DETAILED DESCRIPTION

Mesenchymal stem cells are multipotent cells able to migrate to sites of injury, while also being immunoprivileged by not detectably expressing major histocompatibility complex class II (MHC-II) molecules, and expressing MHC-I molecules at low levels. See Le Blanc, K. et al., Lancet 371 (9624): 1579-1586 (2008) and Klyushnenkova E. et al., J. Biomed. Sci. 12 (1): 47-57 (2005). As such, allogeneic mesenchymal stem cells hold great promise for therapeutic and regenerative medicine, and have been repeatedly shown to have a high safety and efficacy profile in clinical trials for multiple disease processes. See Hare, J. M. et al., Journal of the American College of Cardiology 54 (24): 2277-2286 (2009); Hare, J. M. et al., Tex. Heart Inst. J. 36 (2): 145-147 (2009); and Lalu, M. M. et al., PloS One 7 (10): e47559 (2012). They have also been shown to not undergo malignant transformation after transplantation into patients. See Togel F. et al., American Journal of Physiology Renal Physiology 289 (1): F31-F42 (2005).

Treatment with mesenchymal stem cells has been shown to ameliorate severe graft-versus-host disease, protect against ischemic acute renal failure, contribute to pancreatic islet and renal glomerular repair in diabetes, reverse fulminant hepatic failure, regenerate damaged lung tissue, attenuate sepsis, and reverse remodeling and improve cardiac function after myocardial infarction. See Le Blanc K. et al., Lancet 371 (9624): 1579-1586 (2008); Hare, J. M. et al., Journal of the American College of Cardiology 54 (24): 2277-2286 (2009); Togel F. et al., American Journal of Physiology Renal Physiology 289 (1): F31-F42 (2005); Lee R. H. et al., PNAS 103 (46): 17438-17442 (2006); Parekkadan, B. et al., PloS One 2 (9): e941 (2007); Ishizawa K. et al., FEES Letters 556 (1-3): 249-252 (2004); Nemeth K. et al., Nature Medicine 15 (1): 42-49 (2009); Iso Y. et al., Biochem. Biophys. Res. Comm. 354 (3): 700-706 (2007); Schuleri K. H. et al., Eur. Hearth J. 30 (22): 2722-2732 (2009); and Heldman A. W. et al., JAMA 311 (1): 62-73 (2014). Furthermore, mesenchymal stem cells are also a potential source of multiple cell types for use in tissue engineering (see Gong Z. et al., Methods in Mol. Bio. 698:279-294 (2011); Price, A. P. et al., Tissue Engineering Part A 16 (8): 2581-2591 (2010); and Togel F. et al., Organogenesis 7 (2): 96-100 (2011)).

Mesenchymal stem cells have immuno-modulatory capacity. They control inflammation and the cytokine production of lymphocytes and myeloid-derived immune cells without evidence of immunosuppressive toxicity and are hypo-immunogenic (see Bernardo M. E. et al., Cell Stem Cell 13 (4): 392-402 (2013)).

Mesenchymal stem cells also have the capacity to differentiate not only into cells of mesodermal origin, but into cells of endodermal and ectodermal origin (see Le Blanc K. et al., Exp. Hematol. 31 (10): 890-896 (2003)). For example, in vitro, mesenchymal stem cells cultured in airway growth media differentiate to express lung-specific epithelial markers, e.g., surfactant protein-C, Clara cell secretory protein, and thyroid transcription factor-1 (see Jiang Y. et al., Nature 418 (6893): 41-49 (2002) and Kotton D. N. et al., Development 128 (24): 5181-5188 (2001)).

However, despite being a safe therapeutic agent, mesenchymal stem cells are reported in the literature to exert a suppressive effect on antibody production as well as proliferation and maturation of B cells (see Uccelli, A. et al., Trends in Immunology 28 (5): 219-226 (2007)).

Mesenchymal stem cells are also reported to inhibit the generation and function of antigen presenting cells (see Hoogduijn M. J. et al., Int. Immunopharmacology 10 (12): 1496-1500 (2010)). Finally, mesenchymal stem cells are reported to suppress CD4+ and CD8+ T cell proliferation (see Ghannam S. et al., Stem Cell Res. & Ther. 1:2 (2010)).

Surprisingly, despite the reports of mesenchymal stem cells having a suppressive effect on aspects of the immune system, the present inventors have discovered methods for enhancing a subject's immune response and the performance of their immune system through the administration of a therapeutic amount of bone marrow derived mesenchymal stem cells.

Henceforth, an objective of the present disclosure is to provide methods of treatment or alleviation for aging frailty in subjects in need thereof wherein these methods comprise administering a therapeutic amount of bone marrow derived mesenchymal stem cells to a subject in need.

The methods of treatment described herein can also be used to treat subjects who have frail immune systems from non-aging associated reasons, such as chemotherapy, AIDS/immune-dysfunction, toxin-exposure, Lyme disease, and organ dysfunction/failure. The methods of treatment described herein can also be used to treat subjects who have pre-frail or declining immune systems to prevent them from developing frail immune systems.

In some embodiments, the method of treating or alleviating the symptoms of aging frailty in a subject in need thereof comprises administering a therapeutic amount of LOMECEL-B™ brand isolated allogeneic human stem cells to the subject in need thereof. (LOMECEL-B™ is the brand name for Longeveron, Inc.'s isolated allogeneic human stem cells.)

Further uses and information on the preparation of stem cells that may be suitable for use herein may be found in the following United States Patent Application Publications, all of which are incorporated by reference herein: US2019003874A2; US20190290698A1; and US20200129558A1.

Without being bound to any theory, the bone-marrow derived mesenchymal stem cells can treat or alleviate the symptoms of aging frailty in a subject by either improving the function of endothelial cells, promoting the expression of anti-inflammatory cytokines or cellular pathways, stimulating intrinsic regenerative or repair pathways in neighboring somatic cells or stem cells, improving the function of the immune system, improving the function of the mitochondria in neighboring somatic cells or stem cells, promoting anti-fibrotic pathways or combinations thereof.

In some embodiments, the method further comprises measuring the concentration of a biomarker or biomarkers in the subject suffering from symptoms of aging frailty before and after the administration of a composition comprising a therapeutically effective amount of bone marrow derived MSCs.

The therapeutic amount of bone marrow derived MSCs can range from 25 million cells to 200 million cells. In some embodiments, the therapeutic amount is 25 million cells, 50 million cells, 100 million cells or 200 million cells.

The subjects in need thereof can be any subject suffering from the symptoms of aging frailty, such as geriatric patients or patients over the age of 55, or any subject suffering from symptoms of a frail immune system not originating from aging frailty. In some embodiments the patients are over the age of 65, and in other embodiments they are over the age of 75.

In some embodiments, the method further comprises determining if a change or improvement occurs in a subject's functional mobility and/or exercise tolerance following stem cell administration. The change in a subject's functional mobility and/or exercise tolerance can be determined, for example, by examining whether there is an improvement in a six minute walking distance test by the subject after administration of bone marrow derived MSCs. In these embodiments “improvement” means that the subject is able to walk or travel a farther distance after being administered a therapeutic amount of bone marrow derived MSCs when compared to the distance achieved before administration of the MSCs.

In other embodiments, the method further comprises determining if a change or improvement occurs in a subject's ability to perform activities of daily living. A PROMIS Physical Function score can be used to determine whether a subject's ability to perform activities of daily living has improved. In these embodiments “improvement” means that the subject possess a higher PROMIS Physical Function score after being administered a therapeutic amount of bone marrow derived MSCs when compared to the score achieved before administration of the MSCs.

In some embodiments, the method further comprises determining if a change or improvement occurs in a subject's PROMIS Mobility score. In these embodiments “improvement” means that the subject possess a higher PROMIS Mobility score after being administered a therapeutic amount of bone marrow derived MSCs when compared to the score achieved before administration of the MSCs.

In other embodiments, the method further comprises determining if a change or improvement occurs in a subject's handgrip strength. In these embodiments “improvement” means that the subject is able to hold onto an object longer or tighter after being administered a therapeutic amount of bone marrow derived MSCs when compared to the time or pressure/force achieved before administration of the MSCs.

In some embodiments, the method further comprises determining if a change or improvement occurs in a subject's gait or balance. The Tinetti Performance Oriented Mobility Assessment (POMA) test can be used to determine whether an improvement in a subject's gait or balance has occurred. In these embodiments “improvement” means that the subject is achieves a higher Tinetti POMA score after being administered a therapeutic amount of bone marrow derived MSCs when compared to the score achieved before administration of the MSCs.

Another objective of the present disclosure is to provide novel biomarkers for diagnosing and evaluating the progression of aging frailty in a subject in need thereof. These novel biomarkers can also be measured to determine the effectiveness of the treatment methods described herein.

In some embodiments, the novel biomarkers comprise a change in the concentration levels of pro-inflammatory cytokines within the subject in need thereof. These pro-inflammatory cytokines can be selected from TNF-α, TGF-β, IL-1β, IL-2, D-dimer, C-reactive protein (CRP) or combinations thereof. In preferred embodiments, the concentration of the pro-inflammatory cytokines is decreased in the serum, plasma or blood of the subject in need thereof suffering from aging frailty symptoms after administration of a therapeutic amount of bone marrow derived MSCs to said subject. The pro-inflammatory cytokine concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the pro-inflammatory cytokine concentration is decreased to a stable concentration level wherein the concentration does not increase more than 0% to 10%, 0% to 5% or 0% to 1% once it has reached and maintained a concentration level that is different from the concentration level before administration of bone marrow derived MSCs to the subject in need thereof.

In some embodiments, the novel biomarkers comprise a change in the concentration levels of anti-inflammatory cytokines within the subject in need thereof. These anti-inflammatory cytokines can be selected from IL-8, soluble IL-2 receptor α (sIL-2Rα), IL-4, IL-10, IL-12, TNF-α stimulated gene 6 (TSG-6), or combinations thereof. In preferred embodiments, the concentration of the anti-inflammatory cytokines is increased in the serum, plasma or blood of the subject in need thereof suffering from aging frailty symptoms after administration of a therapeutic amount of bone marrow derived MSCs to said subject. The anti-inflammatory cytokine concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the anti-inflammatory cytokine concentration is increased to a stable concentration level wherein the concentration does not increase more than 0% to 10%, 0% to 5% or 0% to 1% once it has reached and maintained a concentration level that is different from the concentration level before administration of bone marrow derived MSCs to the subject in need thereof.

In other embodiments, the novel biomarkers comprise a change in the concentration of soluble Tie2 (sTie2). Tie2 is receptor tyrosine kinase for angiopoietins and is involved in anti-inflammatory, endothelial integrity and angiogenesis cellular pathways. In inflammatory milieu or cellular environments, Tie2 is proteolytically cleaved to form sTie2, which is typically detected in the serum. sTie2 can inhibit pro-vascular signaling of the full length membrane-bound Tie2. VEGF has also been shown to stimulate Tie2 signaling. (See Singh et al, Cellular Signaling 2009).

Without being bound to any theory, the administration of a therapeutic amount of bone marrow derived MSCs can reduce the concentration of sTie2 in a subject suffering from aging frailty and increase vascular stabilization via Tie2 signaling and VEGF/VEGFR signaling.

In some embodiments, the concentration of sTie2 is decreased in the serum, plasma or blood of the subject in need thereof suffering from aging frailty symptoms after administration of a therapeutic amount of bone marrow derived MSCs to said subject. The sTie2 concentration decrease can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the sTie2 concentration is decreased to a stable concentration level wherein the concentration does not increase more than 0% to 10%, 0% to 5% or 0% to 1% once it has reached and maintained a concentration level that is different from the concentration level before administration of bone marrow derived MSCs to the subject in need thereof.

In other embodiments, the novel biomarkers comprise a change in the concentration levels of VEGF within the subject in need thereof. In preferred embodiments, the concentration of VEGF is increased in the serum, plasma or blood of the subject in need thereof suffering from aging frailty symptoms after administration of a therapeutic amount of bone marrow derived MSCs to said subject. The VEGF concentration increase can range from 0% to 10%, 0.5% to 10%, 1.0% to 10%, 3% to 10%, 5% to 10%, 7% to 10%, greater than 0% to less than or equal to 10%, 10% to 50%, 20% to 50%, 30% to 50% or greater than 50%. In preferred embodiments, the VEGF concentration is increased to a stable concentration level wherein the concentration does not decrease more than 0% to 10%, 0% to 5% or 0% to 1% once it has reached and maintained a concentration level that is different from the concentration level before administration of bone marrow derived MSCs to the subject in need thereof.

### EXAMPLES

### Example 1: A Phase 2b, Randomized, Blinded and Placebo-Controlled Trial to Evaluate the Safety and Efficacy of Lomecel-B Infusion in Patients with Aging Frailty.

### Trial Design:

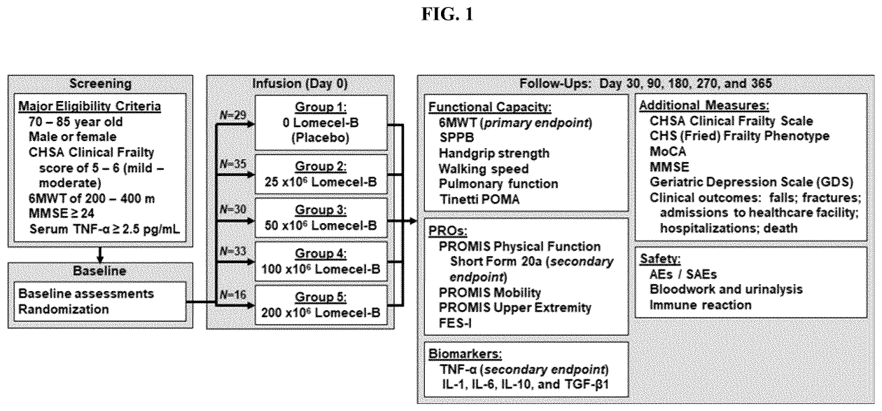

The Phase 2b trial was a multi-site, randomized, double-blinded, placebo-controlled, parallel arm study (FIG. 1). The total duration for each subject after infusion of Lomecel-B cells is 12 months, with up to an additional 2 months for the Screening and Baseline Visits. Baseline demographics of the participants participating in this study can be seen in Table 1 below.

TABLE 1
Baseline Demographics

| Age (Years) | Total (N = 148) |
|---|---|
| Mean (SD) | 76 (3.9) |
| Median | 75 |
| Min, Max | 70, 85 |
| 70 to <75 | 66 (44.6) |
| 75 to 85 | 82 (55.4) |
| sex-n (%) | |
| Male | 88 (59.5) |
| Female | 60 (40.5) |
| Ethnicity-n (%) | |
| Hispanic or Latino | 78 (52.7) |
| Not Hispanic or Latino | 70(47.3) |
| Race-n (%) | |
| American Indian/Alaska Native | 0 (0) |
| Asian | 0 (0) |
| Native Hawaiian or Other Pacific Islander | 0 (0) |
| Black or African American | 6 (4) |
| White | 142 (96) |
| More than One Race | 0 (0) |
| Characteristics | Total (N = 148) |
| Clinical Frailty Score ) [n (%)] | |
| N | 148 |
| 1 (Very fit) | 0 |
| 2 (Well) | 0 |
| 3 (Managing Well) | 0 |
| 4 (Vulnerable) | 0 |
| 5 (Mildly Frail) | 135 (91.2) |
| 6 (Moderately Frail) | 13 (8.8) |
| 7 (Severely Frail) | 0 |
| 8 (Very Severely Frail) | 0 |
| 9 (Terminally III) | 0 |
| Average | 5.1 |
| MMSE (mean ± SD) | 28.8 ± 1.33 |
| 6 MWT (m) (mean ± SD) | 310.8 ± 63.77 |
| FEV1 (L) (mean ± SD) | 2.0 ± 0.69 |

### Lomecel-B and Placebo:

Lomecel-B is a formulation of allogeneic MSCs sourced from healthy young adult donors in compliance with the Codes of Federal Regulations 1271, and culture-expanded using current Good Manufacturing Practices (cGMP) under and an FDA-approved Chemistry, Manufacturing, and Controls (CMC) section of an IND. The placebo consisted of vehicle that Lomecel-B MSCs are resuspended in (PlasmaLyte-A with 1% human serum albumin). Lomecel-B and placebo were prepared in identically-appearing infusion bags bearing identical appearing labels, and delivered via peripheral intravenous infusion in an out-patient setting.

### Clinical Assessments:

Clinical assessments were performed at baseline, Day 90 of treatment, Day 180 of treatment, and Day 270 of treatment.

### Objectives and Endpoints:

Primary Objective: To determine whether Lomecel-B benefits functional mobility and exercise tolerance compared to placebo in patients with Aging Frailty.
Endpoint: Change in 6 minute walking distance at 180 days post-treatment compared to placebo.
Secondary Objectives: (i) to assess the relationship between changes in a physical performance and function-specific Patient-Reported Outcome (PRO), and changes in functional mobility and exercise tolerance and (ii) to assess the relationship between changes in TNF-a, and changes in functional mobility and exercise tolerance.
   Endpoint (i): PROMIS Physical Function Short Form 20a
   Endpoint (ii): Serum TNF-α levels Exploratory Endpoints are provided in Table 2 below.

TABLE 2

| | |
|---|---|
| Physical Function/Performance | Change in grip strength (dynamometer) |
| Assessments | Change in Short Physical Performance |
| | Battery (SPPB) |
| | Change in Forced Expiratory Volume- |
| | 1 second (FEV-1) |
| | Change in walking speed (4-meter gait |
| | speed test) |
| | Change in Performance Oriented |
| | Mobility Assessment (POMA) score |
| Frailty Status | Change in CSHA Clinical Frailty Scale |
| | Change in CHS Frailty Phenotype |
| | (“Fried's” Phenotype) |
| Patient-Reported | Change in PROMIS-Mobility |
| Outcome/Questionnaires | Change in PROMIS-Upper Extremity |
| | Change in Falls Efficacy Scale- |
| | International (FES-I) score |
| | Change in Geriatric Depression Scale- |
| | Short Form (GDS-SF) |
| | Change in Sexual Quality of Life- |
| | Female (SQOL-F) |
| | Change in International Index of |
| | Erectile Function (IIEF) |
| Laboratory Evaluations | Change in inflammatory biomarkers |
| Cognitive Function | (e.g., IL-1, IL-6, TGFβ) |
| | Change in Mini Mental State Exam |
| | (MMSE) score |
| | Change in Montreal Cognitive |
| | Assessment (MoCA) score |

### Statistical Methods:

Primary Endpoint: The primary efficacy endpoint is the change from baseline in 6MWT at Day 180. Each Lomecel-B group was compared to the placebo in pairwise comparisons using a Mixed-Effect Model Repeated Measure (MMRM) method. Four pairwise comparisons used the appropriate simple contrast for Day 180 post-infusion change (primary endpoint) from Baseline:
25 million Lomecel-B formulation vs placebo
50 million Lomecel-B formulation vs placebo
100 million Lomecel-B formulation vs placebo
200 million Lomecel-B formulation vs placebo

As a secondary analysis of the primary efficacy endpoint, the dose response effect was analyzed using the multiple comparisons and modeling approach. To control for the overall type-I error rate, the statistical significance of each comparison was determined by the Hochberg procedure.

Secondary/Exploratory Endpoints: Statistical testing for the key secondary endpoint PROMIS—Physical Function—Short Form 20a and all other secondary/exploratory endpoints performed without adjusting for multiple testing. The exploratory endpoints analyzed using MMRM in a similar way as the primary endpoint.

### Biomarkers:

Assays were performed to determine the change in concentration of Vascular Endothelial Growth Factor (VEGF), TGF-β, TNF-α and sTIE2.

### Physical Function and Performance Assessments:

Physical Function and Performance assessments were determined by examining whether there were changes in grip strength (dynamometers), Short Physical Performance Battery (SPPB) tests, Forced Expiratory Volume—1 second (FEV-1) tests, Performance Oriented Mobility Assessment (POMA) tests, a 4-meter gait speed test (walking speed) and a 6 minute walking distance test.

### Results:

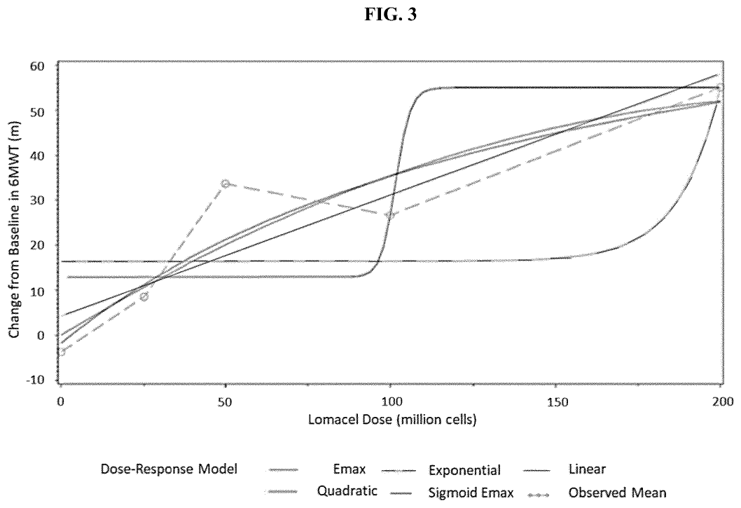

The major new findings of this placebo-controlled trial are that after rigorous examination and evaluation, the trial showed a dose-response (D-R) relationship between the amount of Lomecel-B cells administered and the change in the 6MWT for each cohort (see FIG. 3).

The PROMIS Physical Function PRO scores were highly significantly correlated with the combined Lomecel-B groups. The changes in the PROMIS Physical Function Score and the PROMIS Mobility Score of each cohort can be seen in Tables 3 and 4 below.

TABLE 3
PROMIS Physical Function Score for each Cohort

| | | 25 | 50 | 100 | 200 | | | |
|---|---|---|---|---|---|---|---|---|
| Visit | Statistic | Million | Million | Million | Million | Combined | Placebo | Total |
| Baseline | No. of | 35 | 30 | 33 | 16 | 114 | 29 | 143 |
| Observed | Subjects | | | | | | | |
| Values | | | | | | | | |
| | Mean | 41.44 | 42.89 | 42.74 | 40.91 | 42.12 | 41.42 | 41.98 |
| | Std Dev | 7.390 | 8.750 | 8.461 | 6.046 | 7.863 | 5.679 | 7.459 |
| | Median | 41.10 | 44.00 | 43.90 | 39.85 | 42.05 | 41.30 | 41.60 |
| | Min, Max | 26.5, 62.5 | 27.6, 62.5 | 26.8, 62.5 | 30.5, 52.1 | 26.5, 62.5 | 30.6, 51.2 | 26.5, 62.5 |
| Visit 2, | No. of | 35 | 30 | 33 | 15 | 113 | 29 | 142 |
| Change | Subjects | | | | | | | |
| from BL | | | | | | | | |
| 90 Days | Mean | 2.42 | 3.15 | 2.25 | 0.67 | 2.33 | 1.35 | 2.13 |
| | Std Dev | 7.056 | 7.495 | 5.185 | 3.646 | 6.289 | 4.216 | 5.925 |
| | Median | 3.00 | 2.65 | 2.10 | 0.00 | 2.30 | 1.30 | 1.75 |
| | Min, Max | −11.1, 19.5 | −12.8, 22.4 | −11.5, 13.1 | −5.0, 7.0 | −12.8, 22.4 | −7.7, 11.6 | −12.8, 22.4 |
| | LS Mean | 2.30 | 3.33 | 2.40 | 0.19 | 2.05 | 1.22 | |
| | 95% CI | (0.39, 4.20) | (1.27, 5.39) | (0.44, 4.36) | (−2.70, 3.08) | (0.94, 3.17) | (−0.87, 3.31) | |
| | P-value | 0.0184 | 0.0017 | 0.0169 | 0.8970 | 0.0004 | 0.2512 | |
| Visit 2, vs | P-value | 0.4522 | 0.1574 | 0.4175 | 0.5684 | 0.4875 | | |
| Placebo | | | | | | | | |
| Visit 3, | No. of | 35 | 29 | 31 | 14 | 109 | 28 | 137 |
| Change | Subjects | | | | | | | |
| from BL | | | | | | | | |
| 180 Days | Mean | 1.75 | 2.17 | 3.74 | 1.09 | 2.34 | 2.05 | 2.28 |
| | Std Dev | 7.714 | 5.664 | 5.982 | 4.263 | 6.329 | 4.943 | 6.056 |
| | Median | 2.80 | 3.10 | 2.80 | 0.90 | 2.80 | 1.65 | 2.40 |
| | Min, Max | −13.2, 19.5 | −12.5, 11.1 | −7.8, 18.9 | −5.0, 8.0 | −13.2, 19.5 | −5.6, 9.7 | −13.2, 19.5 |
| | LS Mean | 1.62 | 2.56 | 3.85 | 0.55 | 2.14 | 2.04 | |
| | 95% CI | (−0.39, 3.63) | (0.37, 4.75) | (1.74, 5.96) | (−2.56, 3.66) | (0.95, 3.34) | (−0.19, 4.28) | |
| | P-value | 0.1126 | 0.0224 | 0.0004 | 0.7280 | 0.0005 | 0.0731 | |
| Visit 3, vs | P-value | 0.7820 | 0.7450 | 0.2468 | 0.4416 | 0.9361 | | |
| Placebo | | | | | | | | |
| Visit 4, | No. of | 32 | 30 | 32 | 15 | 109 | 27 | 136 |
| Change | Subjects | | | | | | | |
| from BL | | | | | | | | |
| 270 Days | Mean | 2.89 | 3.32 | 0.57 | 1.07 | 2.08 | 2.16 | 2.09 |
| | Std Dev | 7.766 | 7.667 | 5.758 | 5.709 | 6.948 | 4.990 | 6.589 |
| | Median | 3.85 | 2.25 | 0.00 | 1.50 | 2.10 | 2.40 | 2.10 |
| | Min, Max | −13.4, 21.9 | −11.7, 26.2 | −14.9, 12.6 | −7.6, 10.2 | −14.9, 26.2 | −4.2, 12.6 | −14.9, 26.2 |
| | LS Mean | 2.61 | 3.50 | 0.73 | 0.76 | 1.90 | 2.23 | |
| | 95% CI | (0.45, 4.78) | (1.22, 5.78) | (−1.47, 2.93) | (−2.44, 3.95) | (0.65, 3.15) | (−0.14, 4.61) | |
| | P-value | 0.0182 | 0.0029 | 0.5123 | 0.6413 | 0.0031 | 0.0646 | |
| Visit 4, vs | P-value | 0.8156 | 0.4480 | 0.3598 | 0.4637 | 0.8055 | | |
| Placebo | | | | | | | | |

TABLE 4
PROMIS Mobility Score for each Cohort

| | | 25 | 50 | 100 | 200 | | | |
|---|---|---|---|---|---|---|---|---|
| Visit | Statistic | Million | Million | Million | Million | Combined | Placebo | Total |
| Baseline | No. of | 35 | 30 | 33 | 16 | 114 | 29 | 143 |
| Observed | Subjects | | | | | | | |
| Values | | | | | | | | |
| | Mean | 41.39 | 42.44 | 42.20 | 40.12 | 41.72 | 41.39 | 41.66 |
| | Std Dev | 8.662 | 9.085 | 8.425 | 8.294 | 8.579 | 6.863 | 8.238 |
| | Median | 40.10 | 43.05 | 43.30 | 40.55 | 41.65 | 41.60 | 41.60 |
| | Min. Max | 27.8, 60.1 | 26.7, 60.1 | 27.0, 60.1 | 26.6, 60.1 | 26.6, 60.1 | 28.0, 60.1 | 26.6, 60.1 |
| Visit 2, | No. of | 35 | 30 | 33 | 15 | 113 | 29 | 142 |
| Change | Subjects | | | | | | | |
| from BL | | | | | | | | |
| 90 Days | Mean | 2.06 | 4.29 | 2.07 | 3.01 | 2.78 | 1.38 | 2.49 |
| | Std Dev | 7.599 | 6.993 | 6.204 | 3.413 | 6.601 | 5.222 | 6.352 |
| | Median | 1.50 | 4.05 | 1.70 | 2.70 | 2.50 | 1.00 | 1.95 |
| | Min, Max | −15.2, 17.8 | −11.7, 21.6 | −11.1, 15.7 | −2.1, 9.4 | −15.2, 21.6 | −9.2, 18.5 | −15.2, 21.6 |
| | LS Mean | 2.01 | 4.45 | 2.18 | 2.08 | 2.68 | 1.33 | |
| | 95% CI | (−0.08, 4.09) | (2.20, 6.71) | (0.03, 4.33) | (−1.08, 5.24) | (1.46, 3.91) | (−0.96, 3.62) | |
| | P-value | 0.0590 | 0.0001 | 0.0465 | 0.1951 | 0.0000 | 0.2541 | |
| Visit 2, vs | P-value | 0.6644 | 0.0566 | 0.5908 | 0.7029 | 0.3044 | | |
| Placebo | | | | | | | | |
| Visit 3, | No. of | 35 | 29 | 31 | 14 | 109 | 28 | 137 |
| Change | Subjects | | | | | | | |
| from BL | | | | | | | | |
| 180 Days | Mean | 1.53 | 1.83 | 2.63 | 1.80 | 1.96 | −0.15 | 1.53 |
| | Std Dev | 7.208 | 6.193 | 6.206 | 5.731 | 6.415 | 6.095 | 6.386 |
| | Median | 1.30 | 0.70 | 1.60 | 1.25 | 1.60 | 0.75 | 1.30 |
| | Min, Max | −15.1, 17.8 | −13.0, 14.3 | −11.7, 13.3 | −11.5, 16.1 | −15.1, 17.8 | −15.3, 10.8 | −15.3, 17.8 |
| | LS Mean | 1.47 | 2.13 | 2.60 | 1.44 | 1.91 | −0.13 | |
| | 95% CI | (−0.59, 3.54) | (−0.12, 4.39) | (0.42, 4.77) | (−1.76, 4.64) | (0.68, 3.14) | (−2.42, 2.17) | |
| | P-value | 0.1605 | 0.0634 | 0.0195 | 0.3758 | 0.0026 | 0.9141 | |
| Visit 3, vs | P-value | 0.3079 | 0.1676 | 0.0911 | 0.4339 | 0.1250 | | |
| Placebo | | | | | | | | |
| Visit 4, | No. of | 32 | 30 | 32 | 15 | 109 | 27 | 136 |
| Change | Subjects | | | | | | | |
| from BL | | | | | | | | |
| 270 Days | Mean | 1.53 | 2.71 | 1.29 | 1.55 | 1.79 | 0.81 | 1.59 |
| | Std Dev | 8.045 | 8.138 | 5.527 | 4.311 | 6.919 | 4.913 | 6.565 |
| | Median | 2.35 | 2.10 | 1.00 | 1.90 | 1.80 | 2.20 | 1.80 |
| | Min, Max | −16.1, 17.5 | −12.6, 21.6 | −9.8, 12.8 | −9.2, 8.5 | −16.1, 21.6 | −8.9, 10.8 | −16.1, 21.6 |
| | LS Mean | 1.48 | 2.87 | 1.27 | 1.43 | 1.76 | 0.91 | |
| | 95% CI | (−0.68, 3.64) | (0.60, 5.15) | (−0.92, 3.47) | (−1.77, 4.62) | (0.52, 3.01) | (−1.46, 3.27) | |
| | P-value | 0.1764 | 0.0137 | 0.2529 | 0.3785 | 0.0068 | 0.4490 | |
| Visit 4, vs | P-value | 0.7231 | 0.2387 | 0.8234 | 0.7968 | 0.5278 | | |
| Placebo | | | | | | | | |

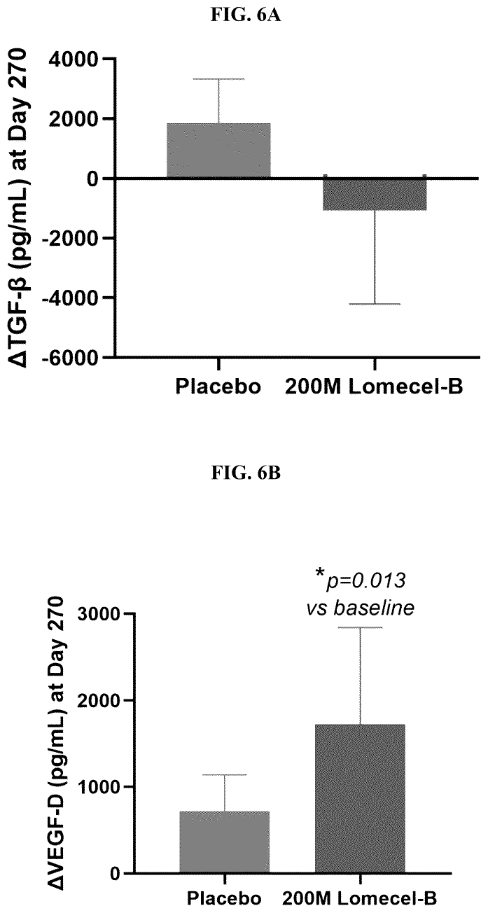

A D-R was also found with other biomarkers. Specifically, as the amount of LOMECEL-B cells administered increased, so did the concentration of Tie2 and VEGF (see FIG. 6B). Furthermore, as the amount of LOMECEL-B cells administered increased, the concentration of TGF-β decreased (see FIG. 6A).

Handgrip strength was shown to increase in the dominant hand for the 100 million cell cohort when compared to both baseline measurements and the placebo cohort. Handgrip strength also increased in the non-dominant hand for the 100 million cell cohort when compared to the placebo cohort. The total balance and total gait, in addition to the derived balance and gait score, significantly increased in the 100 million cell cohort at 270 days.

The trial was shown to be safe and effective with no grade 3 or greater treatment-related adverse effects. The safety results of the trial can be seen in Table 5 below.

TABLE 5
Safety Results

| Adverse Event Category | Total [n (%) m] |
|---|---|
| Any Treatment-Emergent Adverse Events | 65 (43.9) 137 |
| Any Grade 3 or Greater Adverse Events | 10 (6.8) 14 |
| Any Treatment-Related Adverse Events | 5 (3.4) 6 |
| Any Grade 3 or Greater Treatment-Related | 0 |
| Adverse Events | |
| Any Serious Adverse Events | 14 (9.5) 18 |
| Any Treatment-Related Serious Adverse Events | 0 |
| Any Adverse Events with Outcome of Death | 2 (1.4) 4 |
| Adverse Events of Special Interest | 0 |
| Any Adverse Events Leading to | 0 |
| Discontinuation of Treatment | |
| Any Treatment-Related Adverse Events | 0 |
| Leading to Discontinuation of Treatment | |

Overall, the clinical trial demonstrated that administration of bone-marrow derived LOMECEL-B cells to subjects suffering from symptoms of aging frailty can alleviate those symptoms and improve quality of life for those subjects.

### Example 2: A Randomized, Double-Blind and Placebo-Controlled Study to Evaluate the Safety and Efficacy of Lomecel-B Infusion in Patients with Aging Frailty

### Patient Demographics:

Subjects met enrollment criteria of being 70-85 years of age, being cognitively unimpaired (Mini-Mental Scale Exam score >24) and having mild to moderate frailty as assessed by the Canadian Health and Aging Study (CSHA) Clinical Frailty Scale (CFS) (score of 5 or 6, respectively) (see Juma S, Taabazuing MM, Montero-Odasso M. Clinical Frailty Scale in an Acute Medicine Unit: a Simple Tool That Predicts Length of Stay. Canadian geriatrics journal: CGJ 2016; 19:34-9; see Ritt M, Ritt J I, Sieber C C, Gassmann K G. Comparing the predictive accuracy of frailty, comorbidity, and disability for mortality: a 1-year follow-up in patients hospitalized in geriatric wards. Clinical interventions in aging 2017, 12:293-304). In addition, each subject had a screening 6MWT distance between 200 and 400 m (see Cesari M, Bernabei R, Vellas B, et al. Challenges in the Development of Drugs for Sarcopenia and Frailty—Report from the International Conference on Frailty and Sarcopenia Research (ICFSR) Task Force. J Frailty Aging 2022; 11:135-42), and a baseline serum TNF-α≥2.5 pg/ml. Oversight was performed by a single institutional review board (Western IRB: Puyallup, WA), an independent pharmacovigilance group (ProPharma Group: Washington, DC), a Data and Safety Monitoring Board (DSM21B) appointed by the National Institute of Aging (NIA) of the National Institutes of Health (NIH), and independent clinical monitors (Syneos/Joulé Inc.: Edison, NJ). IQVIA (Durham, NC) was the CRO for this study.

Subjects received placebo, or Lomecel-B at doses of 2.5×10⁷ cells (“25M”), 5.0×10⁷ cells (“50M”), 1.0×10⁸ cells (“100M”), or 2.0×10⁸ cells (“200M”). Subjects were initially randomized 1:1:1:1 each to placebo, or 25M, 50M, or 100M Lomecel-B using block sizes of 4. The addition of the 200M Lomecel-B arm was introduced after 92 patients were enrolled. Accordingly, to balance out this new arm with the others within each investigator center, the randomization scheme was modified from central randomization to center-stratified randomization. To maintain the blinding, the treatment allocation was designed such that the “200M” group had the highest chance to be randomized while all other groups still had a chance to be randomized.

### Lomecel-B Cells and Placebo:

Lomecel-B and placebo were manufactured per the Chemistry, Manufacturing, and Controls (CMC) section of an Investigation New Drug Application (IND). Allogeneic MSCs for Lomecel-B were sourced from healthy young adult donors in compliance with the Codes of Federal Regulations 1271, and culture-expanded to high homogeneity using current Good Manufacturing Practices (cGMP). Release criteria included the following: cell viability ≥70%; endotoxin ≤5 EU/mL; mycoplasma negative; USP 71 Sterility negative/no growth; ≥95% positive for CD73, CD90, and CD105 by flow cytometry; ≤2% positive for CD45, CD11b, and CD19; ≤5% positive CD34. Each dose was cryopreserved until needed for infusion.

The placebo was PlasmaLyte-A with 1% human serum albumin, which was the vehicle used for the final formulation of Lomecel-B. Lomecel-B and placebo were delivered via peripheral intravenous infusion at ˜2 mL/min (80 mL total volume administered over ˜40 min) in an outpatient setting. To maintain blinding, Lomecel-B and placebo were prepared in identically appearing infusion bags bearing indistinguishable labels.

### Patient Reported Outcomes (PRO):

The Patient-Reported Outcomes (PRO) Measurement Information System (PROMIS) is a set of NIH-developed validated PROs for evaluating physical, mental, and social health (see Cella D, Yount S, Rothrock N, et al. The Patient-Reported Outcomes Measurement Information System (PROMIS): progress of an NIH Roadmap cooperative group during its first two years. Medical care 2007;45: S3-S11). The adult PROMIS Physical function—Short Form 20a (SF20), used to evaluate patient-reported overall physical functioning, was used as a secondary endpoint since it has been shown to have strong test-retest reliability and a minimally clinically important difference of 2 points (˜0.20 SD). The PROMIS Mobility and PROMIS Upper Extremity were used to evaluate mobility and upper body function, respectively, as pre-specified exploratory endpoints.

### Biomarkers:

Blood collections were performed between 9 and 11 AM to minimize circadian rhythm fluctuations (see Born J, Lange T, Hansen K, Molle M, Fehm H L. Effects of sleep and circadian rhythm on human circulating immune cells. Journal of Immunology 1997; 158:4454-64). Serum and plasma samples were centrifuged on-site shortly after collection, aliquoted, snap-frozen, and cryostored until use. Biomarker analyses were performed double-blinded. To the best extent possible, sample from all time-points for each patient were run in parallel to minimize inter-experimental variability. The central lab was Q² (an IQVIA company; Durham NC), which performed blood and urine safety analyses, and high-sensitivity electrochemiluminescent multiplex immunoassays on serum samples using a Meso QuickPlex system and V-Plex proinflammaory panels K151A9H and K15049D (Meso-Scale Discovery (MSD): Rockville, MD). Longeveron used a Meso QuickPlex system to run the V-Plex Angiogenesis Panel (K15190D).

### Safety Assessments:

Safety assessments included evaluation of adverse events (AEs) and serious AEs (SAEs) for frequency, severity, and blinded relationship to study product. AEs were coded by primary system organ class (SOC) and preferred term (PT) according to the Medical Dictionary for Regulatory Activities (MedDRA) 23.0. The treatment-emergent (TE-) AEs and TE-SAEs were summarized by the number and percentage (n and %) of subjects in each SOC and PT. When multiple AEs were reported with the same preferred term, the AE of the strongest relation is included in summary by relationship, and the AE of the most severe grade is included in the summary by severity table.

### Statistical Analyses:

Unblinding and statistical analyses were performed by an independent statistician group (Pharma Data Associates, LLC: Piscataway, NJ). The sample size was calculated based on the primary endpoint of change from baseline in 6MWT (see Oliva A A, al. e. Results and Insights from a Phase 1 Clinical Trial of Lomecel-B for Alzheimer's Disease. Alzheimer's & Dementia 2022: Accepted). Using a one-sided α=0.025 and effect size of 0.75 (calculated via the difference in change from baseline in 6MWT of the interventional arms versus placebo divided by the common standard deviation of 75 m), 30 subjects per arm provided approximately 80% power for a treatment difference of 56 m. This distance is less than the changes seen in the prior phase 1/2 study (up to 76.6 m) (see Golpanian S, DiFede D L, Khan A, et al. Allogeneic Human Mesenchymal Stem Cell Infusions for Aging Frailty. J Gerontol A Biol Sci Med Sci 2017;72:1505-12; see Tompkins B A, DiFede D L, Khan A, et al. Allogeneic Mesenchymal Stem Cells Ameliorate Aging Frailty: A Phase II Randomized, Double-Blind, Placebo-Controlled Clinical Trial. J Gerontol A Biol Sci Med Sci 2017;72:1513-22).

Efficacy endpoints analyses were performed on the modified intent-to-treat (MITT) population, defined as all randomized subjects who received an infusion and completed at least one post-baseline assessment for the primary efficacy endpoint. Each Lomecel-B group was compared to placebo pairwise using a Mixed-Effect Model Repeated Measure (MMRM), and least squares means (LSM) calculated for comparisons of changes in Lomecel-B groups to changes in placebo. An unstructured variance-covariance matrix was used to model the correlation among repeated measurements. Dose-response effects were calculated using a multiple comparison procedure-modeling (MCP-Mod) method, which is a hybrid approach combining hypothesis testing and modeling to analyze phase 2 dose-ranging studies to find suitable dose(s) for confirmatory phase 3 trials (see Bretz F, Pinheiro J C, Branson M. Combining multiple comparisons and modeling techniques in dose-response studies. Biometrics 2005;61:738-48; see Menon S M, Zink R C. Modern Approaches to Clinical Trials Using SAS: Classical, Adaptive, and Bayesian Methods: SAS Institute; 2105). The candidate models included linear, quadratic, exponential, Emax, and Sigmoid Emax dose-response model. Addition to the adjusted p-values, the Akaike Information Criterion (AIC) was used to evaluate the best parsimonious and predictive model. Smaller AIC means better model. The model mean of the dose-response curve were plotted with 95% confidence intervals. The Safety Population, for evaluating safety, was defined as all subjects who received an infusion.

To account for multiple testing of the different dose groups versus placebo, the step-up Hochberg procedure was used for the primary analysis of the primary endpoint. Secondary analysis of the primary endpoint was dose-response effect via MCP-Mod method.

Simple linear regressions and correlations were calculated for the absolute values and changes from baseline between the 6MWT and patient reported outcome questionnaire.

### Results:

Between August 2017 and February 2020, 365 patients were screened, and 155 met all inclusion/exclusion criteria and were randomized. Reasons for screen failure were TNF-α<2.5 pg/mL (n=57; 26.9%), hepatitis B virus positivity (n=28; 13.2%), 6MWT distance out of range (n=19; 9.0%), HbAlc>8.0% (n=19; 9.0%), and various other reasons (each <9%). Seven subjects were excluded from the analyses due to withdrawal prior to infusion. The remaining 148 subjects received a single infusion of study product (Lomecel-B or placebo), and were included in the safety population analyses. Of these, 137 completed the trial (95.8%), 5 discontinued by choice (3.4%), 4 were lost to follow-up (2.7%), and 2 died on study (1.4%). Of these, 143 had at least 1 follow-up visit, and made up the mITT population for efficacy analysis. Table 6 showed well-balanced characteristics between the 5 groups. The mean participant age was 74.3 to 76.8 years, with 20.6% to 53.3% female, and generally mild frailty with mean CFS score of 5.1.

TABLE 6
Screening/Baseline Demographics (Safety Population; N = 148)

| Treatment group | Placebo | 25M | 50M | 100M | 200M |
|---|---|---|---|---|---|
| Number of subjects (N) | 30 | 37 | 31 | 34 | 16 |
| Age (Mean years ± SD) | 74.3 ± 4.1 | 76.8 ± 3.8 | 74.8 ± 3.7 | 75.8 ± 3.8 | 75.8 ± 4.0 |
| Female sex [n (%)] | 16 (53.3) | 16 (43.2) | 14 (45.2) | 7 (20.6) | 7 (43.8) |
| Ethnicity [n (%)] | | | | | |
| Hispanic/Latino | 15 (50.0) | 21 (56.8) | 14 (45.2) | 23 (67.6) | 5 (31.3) |
| Race [n (%)] | | | | | |
| White | 28 (93.3) | 36 (97.3) | 29 (93.5) | 33 (97.1) | 16 (100.0) |
| Black/African American | 2 (6.7) | 1 (2.7) | 2 (6.5) | 1 (2.9) | 0 |
| 6 MWT | 314.7 ± 64.9 | 304.9 ± 59.0 | 316.8 ± 59.2 | 311.6 ± 55.4 | 308.1 ± 63.1 |
| (meters; mean ± SD) ^{A} | | | | | |
| TNF-α | 3.77 ± 1.19 | 3.90 ± 1.65 | 3.88 ± 0.83 | 3.54 ± 0.98 | 3.79 ± 1.16 |
| (Mean pg/mL ± SD) ^{B} | | | | | |
| PROMIS Physical | 41.4 ± 5.7 | 41.4 ± 7.4 | 42.9 ± 8.8 | 42.7 ± 8.5 | 40.9 ± 6.1 |
| Function SF-20 | | | | | |
| (Mean score ± SD) ^{A} | | | | | |
| PROMIS Mobility | 41.4 ± 6.9 | 41.4 ± 8.7 | 42.4 ± 9.1 | 42.2 ± 8.4 | 40.1 ± 8.3 |
| (Mean score ± SD) ^{A} | | | | | |
| PROMIS Extremity | 45.1 ± 8.6 | 43.5 ± 10.0 | 46.6 ± 11.1 | 45.8 ± 10.1 | 42.3 ± 9.1 |
| (Mean score ± SD) ^{A} | | | | | |
| MMSE | 28.8 ± 1.5 | 28.7 ± 1.4 | 28.8 ± 1.4 | 28.8 ± 1.4 | 29.1 ± 0.8 |
| [Mean points ± SD] | (25-30) | (25-30) | (25-30) | (25-30) | (25-30) |
| TIE2 [Mean ng/L ± SD] ^{A} | 4089.0 ± | 3473.3 ± | 4422.1 ± | 3765.0 ± | 3600.0 ± |
| | 2374.5 | 1372.8 | 1643.9 | 1722.1 | 1675.4 |
| CSHA Clinical Frailty | 5.1 ± 0.3 | 5.1 ± 0.3 | 5.1 ± 0.3 | 5.1 ± 0.3 | 5.1 ± 0.3 |
| Scale (CSF) | | | | | |
| (Mean score ± SD) ^{A} | | | | | |
| CHS Frailty “Fried” | 1.4 ± 1.1 | 1.5 ± 1.1 | 1.4 ± 1.1 | 1.5 ± 1.2 | 1.1 ± 0.9 |
| Phenotype | | | | | |
| (Mean score ± SD) ^{A} | | | | | |

^{A} Efficacy baseline assessment from the mITT population (N = 143).
^{B} TNF-α values are from the screening visit.

### Dose-Dependent Increase in 6MWT

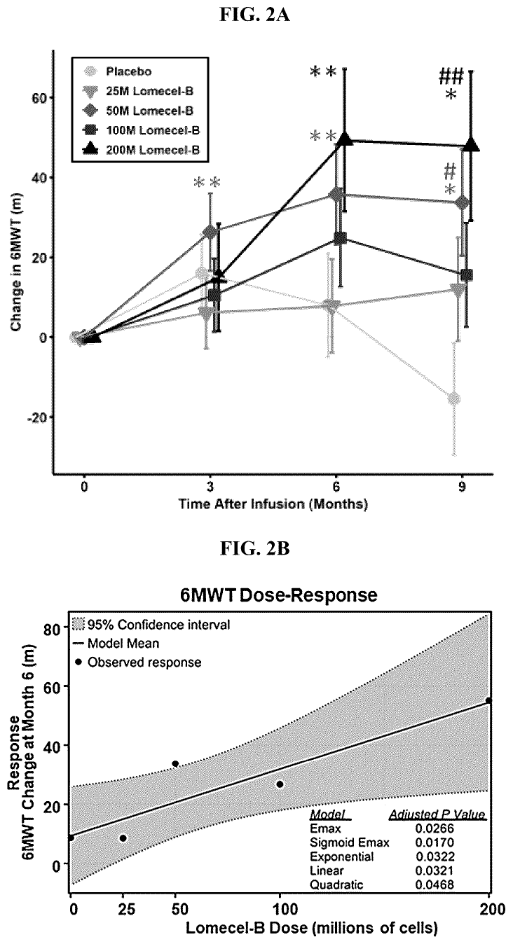

Baseline 6MWT distances were comparable for all arms, approximately 300 meters in all groups (see Table 6). The first component of the primary endpoint was change in 6MWT for an individual dose of Lomecel-B relative to placebo at 6 months post-infusion (see FIG. 2A; Table 7). A formal dose-response analysis, a second, pre-specified component of the primary end-point, showed a statistically significant relationship of increasing Lomecel-B dosage to increased change in 6MWT distance (FIG. 2B). All 5 dose-response candidate models tested were significant (p<0.05 for each), in which the dose-response was best modeled by the linear curve, which has a significant p-value (p=0.0321) and the smallest AIC within the dose-range tested.

TABLE 7
6 MWT Results

| | Placebo | 25M | 50M | 100M | 200M |
|---|---|---|---|---|---|
| Month 3 change | | | | | |
| from baseline | | | | | |
| LS Mean | 16.09 | 6.16 | 26.34 | 10.51 | 14.95 |
| 95% CI | −3.45, 35.62 | −11.60, 23.93 | 7.14, 45.54 | −7.57, 28.60 | −11.60, 41.49 |
| Month 3 change | | | | | |
| versus placebo | | | | | |
| LSM for Difference | | −9.92 | 10.26 | −5.57 | −1.14 |
| 95% CI | | −36.35, 16.50 | −17.11, 37.63 | −32.19, 21.05 | −34.11, 31.83 |
| Month 6 change | | | | | |
| from baseline | | | | | |
| LS Mean | 8.03 | 7.82 | 35.75 | 24.88 | 49.33 |
| 95% CI | −17.64, 33.71 | −15.26, 30.90 | 10.82, 60.67 | 0.64, 49.11 | 14.04, 84.62 |
| Month 6 change | | | | | |
| versus placebo | | | | | |
| LSM for Difference | | −0.21 | 27.71 | 16.84 | 41.29 |
| 95% CI | | −34.75, 34.32 | −8.06, 63.49 | −18.47, 52.15 | −2.35, 84.94 |
| Month 9 change | | | | | |
| from baseline | | | | | |
| LS Mean | −15.48 | 12.04 | 33.75 | 15.55 | 47.88 |
| 95% CI | −43.29, 12.33 | −13.66, 37.73 | 7.39, 60.10 | −10.12, 41.22 | 10.91, 84.85 |
| Month 9 change | | | | | |
| versus placebo | | | | | |
| LSM for Difference | | 27.52 | 49.23 | 31.03 | 63.36 |
| 95% CI | | −10.35, 65.39 | 10.93, 87.53 | −6.81, 68.87 | 17.10, 109.62 |

The difference in 6MWT between the highest dose of Lomecel-B (2×10⁸ cells) and placebo was 41.3 m (95% CI: −2.4−84.9 M p=0.0635) at 6 months. When differences at 9 months were analyzed, the change in Lomecel-B for 50M and 200M doses did reach significance, reflecting ongoing time-dependent separation between active treatment arms and placebo (see FIG. 2A). At Month 9 post-infusion, the difference in change between the 200M Lomecel-B arm and placebo was 63.4 m (95% CI [17.1, 109.6] m; p=0.0077). In addition, the changes in 6MWT from baseline are also presented (see FIG. 2A). Changes from baseline in 6MWT were significant at 6 and 9 months.

### Patient Reported Outcomes (PRO)

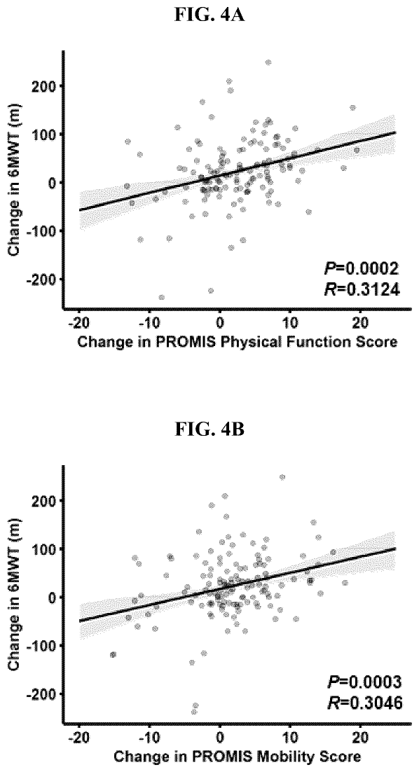

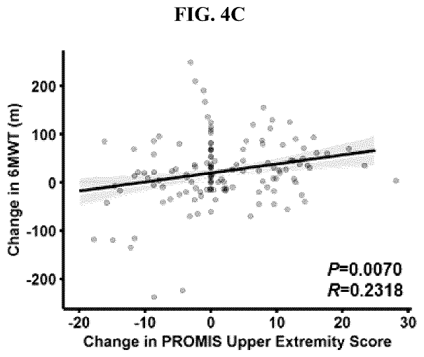

The PROMIS Physical Function SF20 was used as a secondary endpoint to evaluate patient-perceived changes in overall physical functioning. Similarly, the PROMIS Mobility and PROMIS Upper Extremity were used to evaluate mobility and upper body function, respectively, as prospectively tested pre-specified exploratory endpoints. The change in 6MWT and PROMIS Physical Function SF20 were correlated. The Month 6 post-infusion Pearson Correlation Coefficient was 0.3124 (p=0.0002; FIG. 4A). Likewise, the Month 6 post-infusion Correlation Coefficient between the 6MWT and PROMIS Mobility was 0.3046 (p=0.0003; FIG. 4B), and for the 6MWT and PROMIS Upper Extremities was 0.2318 (p=0.0070; FIG. 4C).

sTie2 as a Biomarker of Activity

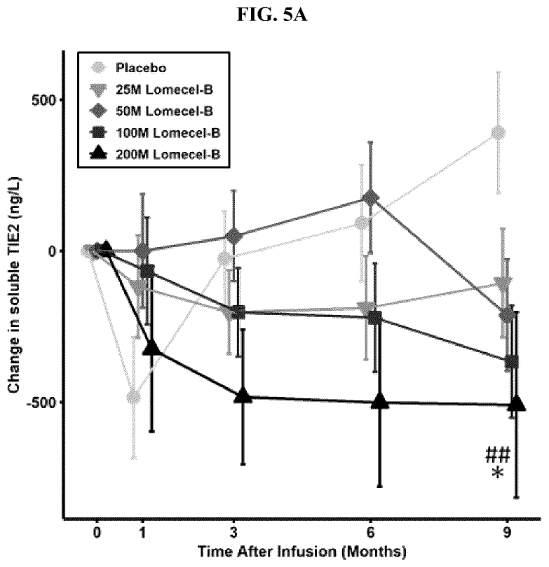

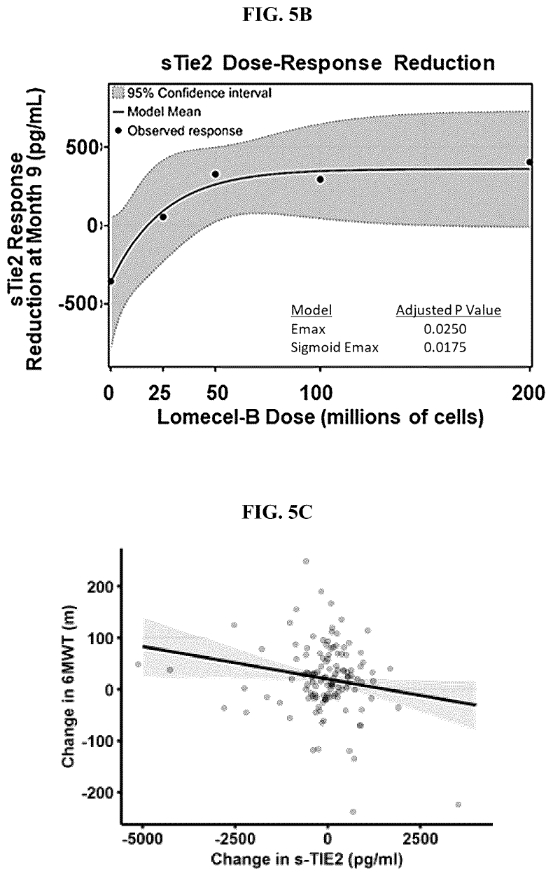

A pre-determined goal of this clinical trial was to identify a biomarker with the potential to predict a functional outcome of Lomecel-B. Among the various potential biomarkers explored in this study, soluble Tie2 (sTie2) was identified as a potential biomarker that met these criteria. sTie2 decreased in the 200M Lomecel-B group (see FIG. 5A) which was significantly different from baseline at 3 months post-infusion (−484.1 pg/mL; 95% CI [−925.33, −42.90] pg/mL; p=0.0318). At 9 months post-infusion, the 200M Lomecel-B group was different from placebo by −936.9 pg/mL (95% CI [−1640.3, −233.4] pg/mL; p=0.0095), as was the 50M Lomecel-B (−601.2 pg/mL; 95% CI [−1137.2, −65.2] pg/mL; p=0.0283) and 100M Lomecel-B groups (−755.4 pg/mL; 95% CI [−1294.1, −216.8] pg/mL; p=0.0064). Dose-response analyses showed best-fit modeling to the Sigmoidal Emax (p=0.0175), in which there appeared to be a plateau at the 50M Lomecel-B dose (see FIG. 5B). Furthermore, the change in sTie2 correlated to the change in 6MWT (r=−0.1850; p=0.0397) (see FIG. 5C).

### Safety and Clinical Events

No safety concerns on this study were raised by either the NIA-appointed DSMB or pharmacovigilance monitor. Overall, the proportion of subjects with TE-SAEs were comparable across the different study arms (see Table 8). There were 2 deaths on study: a pulmonary embolism occurring 296 days post-infusion in the 100M Lomecel-B arm; and a combined cerebral arteriosclerosis/coronary artery disease/aspiration pneumonia occurring 167 days post-infusion in the placebo arm. There were no SAEs attributed to the study product. Two infusions were temporarily interrupted (both in the 25M Lomecel-B arm), but continued to completion, and both subjects completed follow-up visits. All AEs that occurred during the infusion were considered by investigators and DSMB as not product related. There were no statistically significant differences in the rates of falls, fractures, hospitalizations, and admissions to healthcare facilities in any of the Lomecel-B arms versus placebo.

TABLE 8
Safety Summary

| | Placebo | 25M | 50M | 100M | 200M |
|---|---|---|---|---|---|
| | (N = 30) | (N = 37) | (N = 31) | (N = 34) | (N = 16) |
| Subjects with ≥1 TE-SAE [n (%)] | 3 (10.0) | 4 (10.8) | 2 (6.5) | 5 (14.7) | 0 (0.0) |
| Subjects with ≥1 TE-AE [n (%)] | 13 (43.3) | 19 (51.4) | 19 (61.3) | 17 (50.0) | 12 (75.0) |
| Subjects with Grade 3 or Higher | 3 (10.0) | 3 (8.1) | 2 (6.5) | 2 (5.9) | 0 (0.0) |
| TE-AE [n (%)] | | | | | |
| Subjects with ≥1 severe/life- | 3 (10.0) | 3 (8.1) | 2 (6.5) | 2 (5.9) | 0 (0.0) |
| threatening/death TE-AE [n (%)] | | | | | |
| Deaths on study [n (%)] | 1 (3.3) | 0 (0.0) | 0 (0.0) | 1 (2.9) | 0 (0.0) |
| Subjects with infusion-product- | 0 (0.0) | 0 (0.0) | 0 (0.0) | 0 (0.0) | 0 (0.0) |
| related TE-SAE [n (%)] | | | | | |
| Subjects with 1 infusion | 1 (3.3) | 2 (5.4) | 2 (6.5) | 1 (2.9) | 3 (18.8) |
| procedure-related TE-AE [n (%)] | | | | | |
| Subjects discontinuing study due | 0 (0.0) | 0 (0.0) | 0 (0.0) | 0 (0.0) | 0 (0.0) |
| to ≥1 TE-AE [n (%)] | | | | | |
| Subjects with discontinued | 0 (0.0) | 0 (0.0) | 0 (0.0) | 0 (0.0) | 0 (0.0) |
| infusion [n (%)] | | | | | |
| Subjects having falls [n (%)] | 1 (3.4) | 0 | 3 (10.0) | 0 | 0 |
| Subjects having fractures [n (%)] | 0 | 0 | 2 (6.7) | 0 | 1 (6.3) |
| Subjects admitted to healthcare | 1 (3.4) | 3 (8.3) | 1 (3.3) | 4 (12.1) | 0 |
| facility [n (%)] | | | | | |
| Subjects hospitalized [n (%)] | 1 (3.4) | 3 (8.3) | 1 (3.3) | 4 (12.1) | 0 |

One of the major new findings of this study is that a single infusion of Lomecel-B, when compared to placebo, led to a dose-dependent increase in walk distance in older adults with mild to moderate frailty. The increases seen in the highest dose groups exceeded minimally significant clinical thresholds (see Shoemaker M J, Curtis A B, Vangsnes E, Dickinson M G. Clinically meaningful change estimates for the six-minute walk test and daily activity in individuals with chronic heart failure. Cardiopulmonary physical therapy journal 2013;24:21-9; see Kwok B C, Pua Y H, Mamun K, Wong W P. The minimal clinically important difference of six-minute walk in Asian older adults. BMC geriatrics 2013;13:23; see Perera S, Mody S H, Woodman R C, Studenski S A. Meaningful change and responsiveness in common physical performance measures in older adults. Journal of the American Geriatrics Society 2006;54:743-9) and correlated with improvements in patient self-reported outcomes. In addition, serum levels of sTie2, a factor associated with impaired vascular function, also improved (decreased levels) in a dose-dependent fashion to Lomecel-B. These findings are consistent with the potential that Lomecel-B may treat frailty by improving patient's quality of life, improving mobility, and reducing dependency on others.

These findings are also consistent with previous studies in patients with frailty, in which a single infusion of allogeneic MSCs suggested improved walk distance (see Golpanian S et al. 2017; see Tompkins et al. 2017). An additional pre-specified objective of this trial was to establish whether Lomecel-B manifests a dose-response effect. Indeed, a clear and significant dose-response relationship to increase in 6MWT was evident at 6 months post-treatment, supporting evidence of bioactivity. Furthermore, at 9 months after the single infusion, improvement continued to be sustained and reached significance versus placebo, which had begun to decline from baseline. The stronger difference between the treatment and placebo groups observed at 9 months may indicate ongoing and sustained bioactivity, and suggest that future studies should monitor response to treatment for longer than 6 months.

Our findings are consistent with previous studies in patients with frailty, in which a single infusion of allogeneic MSCs suggested improved walk distance (see Golpanian S et al. 2017; see Tompkins et al. 2017). An additional pre-specified objective of this trial was to establish whether Lomecel-B manifests a dose-response effect. Indeed, a clear and significant dose-response relationship to increase in 6MWT was evident at 6 months post-treatment, supporting evidence of bioactivity. Furthermore, at 9 months after the single infusion, improvement continued to be sustained and reached significance versus placebo, which had begun to decline from baseline. The stronger difference between the treatment and placebo groups observed at 9 months may indicate ongoing and sustained bioactivity, and suggest that future studies should monitor response to treatment for longer than 6 months.

PROMIS measures offer insights into the effects of frailty on function and mobility, which are critical for patient-centered clinical decisions. As part of this study, we sought to determine a measure of patient perception of improved physical functioning. Indeed, the PROMIS Physical Function SF20, an assessment of overall physical functioning, showed improvement that significantly correlated with increased 6MWT distance (see FIG. 4A). Moreover, the PROMIS Mobility showed an even stronger significant correlation (see FIG. 4B). These results indicate that the positive effects of Lomecel-B were not just limited to objective physical measures, but generalized to older individuals' perceptions of functional improvement.

Another goal of this study was to identify possible biomarkers that correlated with bioactivity. Among a panel of potential biomarkers, we found that sTIE2 levels were more closely associated with Lomecel-B infusion in a dose-response fashion. TIE-2 is receptor tyrosine kinase present on microvascular endothelium and endothelial precursor cells, and is activated through binding of the angiopoietins, Ang1 and Ang2 (see Sack K D, Kellum J A, Parikh S M. The Angiopoietin-Tie2 Pathway in Critical Illness. Crit Care Clin 2020;36:201-16). Cleavage of the extracellular domain of Tie2 (sTie2), mediated by matrix-metalloprotease (MMP) 14 (see Idowu T O, Etzrodt V, Seeliger B, et al. Identification of specific Tie2 cleavage sites and therapeutic modulation in experimental sepsis. eLife 2020;9), can be detected in the serum, in which increasing levels are indicative of endothelial dysfunction. In the context of Lomecel-B mechanisms of action, the decreased levels of sTie2 support a pro-vascular activity in frailty, and is consistent with the suggestive pro-vascular activity of Lomecel-B for Alzheimer's disease²⁵. The prevention of Tie2 cleavage is a biologically plausible action of Lomecel-B, as MSCs are known to secrete high levels of tissue inhibitors of MMPs (TIMPs) (see Lozito T P, Jackson W M, Nesti L J, Tuan R S. Human mesenchymal stem cells generate a distinct pericellular zone of MMP activities via binding of MMPs and secretion of high levels of TIMPs. Matrix Biol 2014;34:132-43). Future studies are needed to confirm these findings, including determinations of other vascular biomarkers and measures of endothelial function.

In summary, the results of the study indicate that aging-related frailty may respond to infusion of Lomecel-B and lead to clinically meaningful dose-dependent improvements in 6MWT that showed correlations to physical function PROs, and that also revealed a potential dose-dependency to a mechanistically relevant biomarker, sTie2. These data support the advancement of Lomecel-B as a potential treatment for the unmet medical need of frailty. Furthermore, this trial provides a clear demonstration of a dose-dependent relationship of a cell-based therapy providing evidence of bioactivity.

1. A method for alleviating the symptoms of aging frailty in a subject in need thereof, wherein the method comprises administering a composition comprising a therapeutically effective amount of bone marrow derived mesenchymal stem cells (MSCs) to the subject.

2. A method for treating aging frailty or inhibiting aging frailty disease progression, wherein the method comprises administering a composition comprising a therapeutically effective amount of allogeneic mesenchymal stem cells (MSCs) to the subject.

3. The method according to claim 1, wherein the method further comprises determining if a change occurs in the concentration of biomarkers in the subject suffering from symptoms of aging frailty before and after the administration of the composition comprising bone marrow derived MSCs.

4. The method according to claim 3, wherein the method further comprises determining if an improvement occurs in the subject's functional mobility and/or exercise tolerance.

5. The method according to claim 3, wherein the method further comprises determining if an improvement occurs in the subject's ability to perform activities of daily living.

6. The method according to claim 5, wherein determining the improvement in the subject's ability to perform activities of daily living comprises examining the subject's PROMIS Physical Function score before and after administration of the bone marrow derived MSCs.

7. The method according to claim 3, wherein the method further comprises determining if an improvement occurs in the subject's PROMIS Mobility score.

8. The method according to claim 3, wherein the method further comprises determining if an improvement occurs in the subject's handgrip strength.

9. The method according to claim 3, wherein the method further comprises determining if an improvement occurs in the subject's gait or balance.

10. The method according to claim 3, wherein the biomarkers comprise pro-inflammatory cytokines.

11. The method according to claim 10, wherein the pro-inflammatory cytokines are TNF-α, TGF-β, IL-1β, IL-2, D-dimer, C-reactive protein (CRP) or combinations thereof.

12. The method according to claim 9, wherein the concentration of pro-inflammatory cytokines is decreased from 0.5% to 10%, 5% to 10%, 10% to 50%, or greater than 50% after the administration of the composition comprising bone marrow derived MSCs.

13. The method according to claim 3, wherein the biomarkers further comprise anti-inflammatory cytokines.

14. The method according to claim 13, wherein the anti-inflammatory cytokines are IL-8, soluble IL-2 receptor α (sIL-2Rα), IL-4, IL-10, IL-12, TNF-α stimulated gene 6 (TSG-6) or combinations thereof.

15. The method according to claim 13, wherein the concentration of anti-inflammatory cytokines is increased from 0.5% to 10%, 5% to 10%, 10% to 50%, or greater than 50% after the administration of the composition comprising bone marrow derived MSCs.

16. The method according to claim 3, wherein the biomarkers further comprise soluble Tie2 (sTie2).

17. The method according to claim 16, wherein the concentration of sTie2 is decreased from 0.5% to 10%, 5% to 10%, 10% to 50%, or greater than 50% after the administration of the composition comprising bone marrow derived MSCs.

18. The method according to claim 3, wherein the biomarkers further comprise Tie2.

19. The method according to claim 18, wherein the concentration of Tie2 is increased from 0.5% to 10%, 5% to 10%, 10% to 50%, or greater than 50% after the administration of the composition comprising bone marrow derived MSCs.

20. The method according to claim 3, wherein the biomarkers further comprises VEGF.

21. The method according to claim 20, wherein the concentration of VEGF is increased from 0.5% to 10%, 5% to 10%, 10% to 50%, or greater than 50% after the administration of the composition comprising bone marrow derived MSCs.
